(19) United States
(12) Patent Application Publication
(10) Pub. No.: US 2026/0265727 A1
(43) Pub. Date: Sep. 10, 2026

(54) NUCLEIC ACID PRODUCT AND PROCESS

(71) Applicant: UNIBIO A/S, Roskilde (DK)

(72) Inventors: Theis Sommer, Roskilde (DK); Jesper Gebauer, Roskilde (DK); Michael Elleskov, Roskilde (DK); Ida-Maria Heberg Thylkjær, Roskilde (DK); Michael Jensen, Roskilde (DK); Eleni Ntokou, Roskilde (DK)

(21) Appl. No.: 18/875,557

(22) PCT Filed: Jun. 15, 2023
(86) PCT No.: PCT/EP2023/066038

§ 371 Date: Dec. 16, 2024

(30) Foreign Application Priority Data
Jun. 17, 2022 (EP) 22179684.0

(51) Int. Cl. C12N 15/10; C12N 1/00; C12N 1/20; C12R 1/05; C12R 1/07

(52) U.S. Cl. CPC C12N 15/1017; C12N 1/005; C12N 1/20; C12R 2001/05; C12R 2001/07

(57) ABSTRACT

A method is provided for isolating a nucleic acid rich product from an aqueous biomass material. After steps of heat-shock treatment and incubation, the aqueous biomass material is separated, to provide a first concentrated biomass material and a first liquid fraction. Various purification steps are carried out, to make best use of all products in the biomass.

### TECHNICAL FIELD

The present invention relates to a method for isolating a nucleic acid rich product from an aqueous biomass material. After steps of heat-shock treatment and incubation, the aqueous biomass material is separated, to provide a first concentrated biomass material and a first liquid fraction. Various purification steps (e.g. nanofiltration) may then be carried out.

### BACKGROUND

The methanotrophic bacterium Methylococcus capsulatus is a non-commensal bacterium found ubiquitously in nature. It metabolizes methane, e.g., from natural gas, into biomass, CO₂ and water. Being rich in protein, M. capsulatus can be used as a protein supplement in animal feed and is also of interest for human consumption. The fermentation of this bacterium as a protein source for both animal and human consumption may contribute to satisfying the world's need for dietary protein in a way which is more environmentally friendly than conventional protein production industries.

In order to make an industrial process feasible both economically and environmentally, as many valuable products as possible should be isolated, e.g. from waste streams.

WO 2018/115042 relates to a method for removing nucleic acids from a biomass. The methods described therein, however, cannot provide clearly separated fractions, due to complex formation in the disrupted biomass material, in which protein and nucleic acid components are strongly bound.

The heat-shock process described in L. Jørgensen et al (J. Larsen & L. Jørgensen, Applied Microbiology and Biotechnology volume 45, pages 137-140 (1996)) describes how to decrease nucleic acid content in a protein product from a culture of Methylococcus Capsulatus. However, this document does not describe any use or fractionation of waste streams from this heat-shock process.

Related publications include WO2021/071895 and WO2004/029076.

It is an object to improve the utilisation of waste streams from the production of biomass in this manner.

### SUMMARY

It has surprisingly been found that waste streams from the production of biomass contain useful levels of by-product, in particular nucleic acids, which can be readily isolated from the process. It has also been discovered that separation of biomass into protein fractions and nucleic acid fractions can be enabled by pre-treatment of the biomass.

So, in a first aspect the present invention relates to a method for isolating a nucleic acid rich product from an aqueous biomass material, said method comprising the steps of:
subjecting the aqueous biomass material to a heat-shock treatment to provide a heat-shock treated biomass material;
incubating the heat-shock treated biomass material at an elevated temperature for a predetermined time period;
separating the aqueous biomass material in a first separation process, to provide a first concentrated biomass material and a first liquid fraction;
followed by:
   subjecting the first liquid fraction from the first separation process to a first nanofiltration (NF) step, to provide a mineral-rich first NF permeate, and a nucleic acid and peptide-rich first NF retentate in which the nucleic acids are dissolved;
   or
   subjecting the first liquid fraction from the first separation process to a second filtration step, to provide a mineral-rich second permeate, and a nucleic acid-rich second retentate in which the nucleic acids are dissolved;
   followed by:
      subjecting the nucleic acid-rich second retentate from the second filtration step to spraydrying to obtain a nucleic acid-rich product;
      or
      subjecting the nucleic acid-rich second retentate from the second filtration step to a second nanofiltration (NF) step, to obtain a nucleic acid and peptide-rich second NF-retentate and a mineral rich second NF-permeate;
      or
      subjecting the mineral-rich second permeate from the second filtration step to a third nanofiltration step to obtain an RNA rich third NF-retentate and mineral rich third NF-permeate.

This, and other aspects of the invention, are set out in the following patent claims, figures and examples.

### LEGENDS TO THE FIGURES

FIG. 1a shows an overview of the process of the invention, up to the point of the first separation process.

FIG. 1b shows an overview of one option (option 1b) of the process of the invention carried out on the supernatant/permeate (i.e. the liquid fraction) from the first separation process in FIG. 1a, where this is upconcentrated in a first nanofiltration treatment into a nucleic acid (NA) and peptide rich fraction (first NF Retentate) and a first NF-permeate fraction containing minerals and water.

FIG. 1c shows an overview of the process of the invention carried out on the pellet/retentate from the first separation process in FIG. 1a.

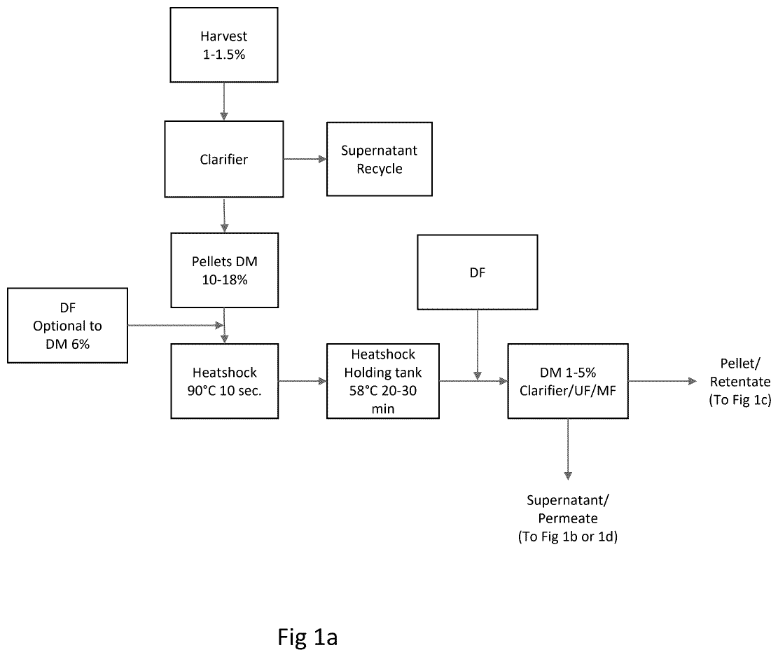

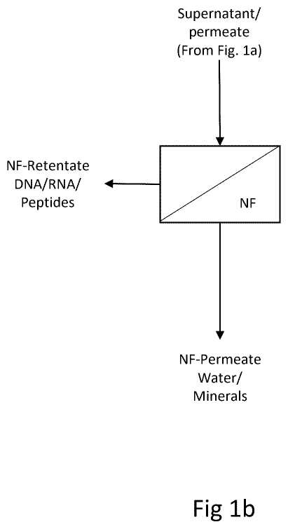

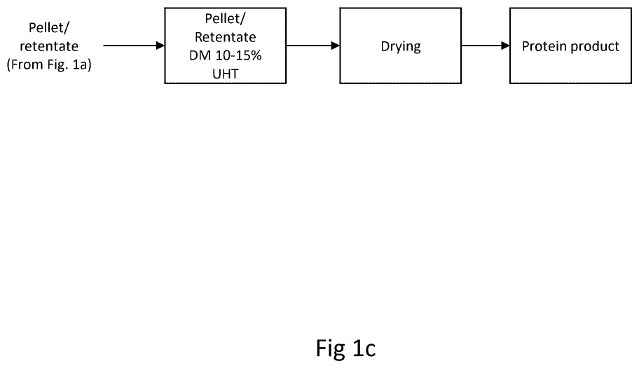

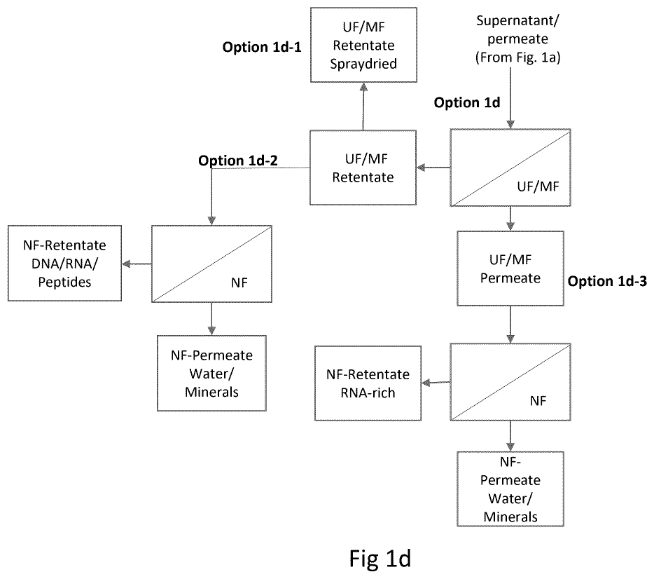

FIG. 1d shows an overview of a second option (option 1d) of the process of the invention carried out on the supernatant/permeate from the first separation process in FIG. 1a, where this is taken to a second filtration step (being MF/UF), to give a mineral-rich second permeate, and a nucleic acid-rich second retentate in which the nucleic acids are dissolved. There are subsequently 3 options:
Option 1d-1: The MF/UF second retentate can be taken to a spray dryer directly
Option 1d-2: The MF/UF second retentate is taken to a second NF-separation step, to provide a nucleic acid and peptide-rich second NF-retentate and a mineral rich second NF-permeate.
Option 1d-3: The MF/UF second permeate is taken to a third nanofiltration unit, where the third NF-retentate is rich in RNA and the third NF-permeate contains only minerals and water.

### DETAILED DISCLOSURE

Throughout this text, the abbreviation “DM” refers to “Dry Matter”. In the present context, the terms “dry matter” and “ash” content is determined according to the A.O.A.C. method (reference A.O.A.C. Standard, 1945).

As used herein, the term “dry weight”, in the context of the dry weight of an M. capsulatus biomass, should be taken to mean the weight of the biomass after all water has been removed from it. This should not be taken to mean that all water has been removed in all embodiments of an M. capsulatus biomass according to the present invention, since water is present in some embodiments. It should rather be understood as a measure that can be used to reproducibly calculate whether a certain biomass falls within the scope of the biomass according to the present invention.

The term “nucleic acids”, as used herein includes DNA (deoxyribonucleic acid) and RNA (ribonucleic acid), as well as fragments thereof. Nucleic acids are made from monomers known as nucleotides.

When a product, such as a permeate or retentate, is described as being “rich” in a certain component after a given process step, it is to be understood that it is “enriched” with said component, compared to the components prior to said process step. For instance, when the first liquid fraction is subjected to a second filtration step, to provide a mineral-rich second permeate, and a nucleic acid-rich second retentate in which the nucleic acids are dissolved, the mineral content of the second permeate is enriched (=increased) relative to the first liquid fraction.

“Nucleic acid rich” or “nucleic acid enriched” means that the ratio of nucleic acid to minerals, protein or dry matter has increased, e.g. increased by at least 30%, during the relevant process step.

Microfiltration is a membrane filtration with membrane pore sizes of >0.1 μm and molecular cut-off values (MWCO)>100 kDa

Ultrafiltration is a membrane filtration with membrane pore sizes of 10 nm to 100 nm and molecular cut-off values (MWCO) 1 kDa to 500 kDa.

Nanofiltration is a membrane filtration with membrane pore sizes of 0.1 nm-20 nm and molecular cut-off values of (MWCO) 0.01 kDa to 10 kDa.

As noted, the invention provides a method for isolating a nucleic acid rich product from an aqueous biomass material.

### Fermentation

The aqueous biomass material is suitably obtained from the fermentation of at least one microorganism, preferably wherein at least one of the microorganisms is a bacterial cell, preferably a methanotroph, more preferably Methylococcus capsulatus.

In a fermentation step, the microorganism, or mixture of microorganisms, metabolizes methane into aqueous biomass material, CO₂ and water. The fermentation occurs in fermentation tanks, and the process is described in detail in, e.g., WO 2017/080987 and WO 2022/008478 hereby incorporated by reference.

The biomass material is a single-cell protein (SCP) product. It comprises a majority of protein (ca. 60%), and lesser amounts of RNA and DNA. When isolated from the fermentation step, the biomass material is an aqueous suspension. In this aqueous suspension, the majority of the solid component is cellular material from the microorganism. Other components (e.g., proteins, nucleic acids, polysaccharides, lipids or other small molecules) may be dissolved or suspended in the aqueous phase.

At least one of the microorganisms used in the fermentation step is suitably a bacterial cell, preferably a methanotroph, more preferably Methylococcus capsulatus. Therefore, the biomass is suitably Methylococcus capsulatus biomass.

The term “Methylococcus capsulatus” or “M. capsulatus”, as used herein, can mean any strain of bacteria belonging to the M. capsulatus species. The strain may be either naturally occurring or developed in a laboratory, such as a genetically modified strain. The term “naturally occurring” means that the strain has not been genetically modified using genetic engineering techniques. However, it may contain natural modifications or alterations in its genetic material compared to a reference strain, such as alterations that occur randomly during replication. Preferably, the strain is naturally occurring. Also preferably, the strain is M. capsulatus (Bath), more preferably the M. capsulatus (Bath) identified under NCIMB 11132. However, it may also be M. capsulatus (Texas) or M. capsulatus (Aberdeen) or a different M. capsulatus strain which is currently known or will be discovered or characterized in the future.

The methanotrophic bacteria may be provided in a co-fermentation together with one or more heterotrophic bacteria. The following heterotrophic bacteria may be particularly useful to co-ferment with M. capsulatus; Ralstonia sp.; Bacillus brevis; Brevibacillus agri; Alcaligenes acidovorans; Aneurinibacillus danicus and Bacillus firmus. Suitable yeasts may be selected from species of Saccharomyces and/or Candida. The preferred heterotrophic bacteria are chosen from Alcaligenes acidovorans (NCIMB 13287), Aneurinibacillus danicus (NCIMB 13288) and Bacillus firmus (NCIMB 13289) and combinations thereof. The methanotrophic bacteria and/or the heterotrophic bacteria may be genetically modified.

In the fermentation step, the carbon source is converted by the microorganism(s) to biomass material. Suitably, the carbon source comprises methane, and is e.g., natural gas, syngas or biogas. During the fermentation step, the carbon source is dissolved in the fermentation medium. Fermentation suitably takes place in a U-loop reactor, as described in WO 2010/069313, hereby incorporated by reference. A suitable fermentation medium is described in e.g. WO 2018/158322 hereby incorporated by reference. The fermentation step has a relatively low Dry Matter content, e.g. below 5%.

The co-fermentation of M. capsulatus with one or more other organisms may result in a biomass product containing an M. capsulatus biomass as well as a biomass of the one or more other organisms.

In some embodiments, the M. capsulatus is fermented in combination with one or more bacteria selected from: Ralstonia sp., B. brevis, B. agri, A. acidovorans, A. danicus, and B. firmus; preferably any one or two or all three of: A. acidovorans, A. danicus, and B. firmus; more preferably any one or two or all three of: A. acidovorans (NCIMB 13287), A. danicus (NCIMB 13288), and B. firmus (NCIMB 13289).

Further details of the fermentation process are described in WO 2020/245197 and WO 2020/249670, which are hereby incorporated by reference.

The dry matter content of the biomass material from the fermentation process is between 1-1.5%. After harvesting the biomass material from the fermentation process, the biomass material may be clarified, and the supernatant from the clarification step may be recycled to the fermenter. A pellet thus obtained is with a dry matter content of 10-18%. The fermentation broth in the fermenter may preferably continuously be provided with the required amounts of water and nutrient salts, such as ammonium/ammonia, magnesium, calcium, potassium, iron, copper, zinc, manganese, nickel, cobalt and molybdenum in the form of sulphates, chlorides or nitrates, phosphates and pH controlling components, i.e. acids and/or bases, as normally used by the skilled person, e.g. sulphuric acid (H₂SO₄), nitric acid (HNO₃), sodium hydroxide (NaOH), potassium nitrate (KNO₃). The latter is also a suitable nitrogen source for M. capsulatus. The specific details of the fermentation process, and substrates etc. is described in WO 2000/70014 and WO 2010/069313, which are incorporated by reference.

The biomass material produced from fermentation of natural gas will comprise from 60 to 80% by weight crude protein; from 5 to 20% by weight crude fat; from 3 to 12% by weight ash; from 3 to 15% by weight nucleic acids (RNA and DNA).

Optionally, the biomass material is subjected to a dilution step at this point, to lower the dry matter content to around 6%.

### Heat Shock

In a first step, the aqueous biomass material is subjected to a heat-shock treatment to provide a heat-shock treated biomass material. The heat-shock treatment is thought to activate endogenous nucleases, to thereby reduce RNA and DNA.

Suitably, the heat-shock treatment takes place at a temperature of at least 70° C., preferably at least 80° C. and more preferably at least 90° C. The heat-shock treatment may take place for a time of between 1 and 200 seconds, preferably between 5 and 100 seconds and more preferably between 10 and 50 seconds. In a preferred embodiment, heat-shock treatment takes place at around 90° C. for a time period of around 10 seconds. Heat-shock treatment may take place by passing the aqueous biomass material through a heated coil.

### Incubation

The heat-shock treated biomass material is then incubated at an elevated temperature for a predetermined time period. During incubation, breakdown of larger RNA and DNA molecules takes place. Incubation of the heat-shock treated biomass material suitably takes place at a temperature of at least 40° C., preferably at least 50° C. and more preferably at least 60° C. Incubation of the heat-shock treated biomass material may take place for a time of between 1 and 200 minutes, preferably between 5 and 100 minutes and more preferably between 10 and 50 minutes. In a preferred embodiment, incubation takes place at between 5° and 60° C. for a time period of 20-30 minutes.

After incubation, the aqueous biomass material typically has a protein content of 50-80% w/w and a nucleic acid content of 4-12% w/w.

### First Separation Process

Following heat-shock and incubation, the aqueous biomass material is subjected to a first separation process, to provide a first concentrated biomass material and a first liquid fraction.

The first separation process may be an ultrafiltration (UF) process, a microfiltration (MF) process or a centrifugation process, and is preferably a UF process. If a MF process is used, it typically has a MCWO of 100-2000 kDa. If a UF process is used, it typically has a MWCO of 10-100 kDa.

If the first separation process is UF, the UF permeate is the first liquid fraction, while the UF retentate is the first concentrated biomass material.

The aqueous biomass material may be washed and diluted prior to the first separation process.

If the first separation process is a centrifugation process, the first liquid fraction is supernatant, while the first concentrated biomass material is a pellet.

The first liquid fraction comprises nucleic acids, peptides, vitamins, minerals and amino acids. The first separation process may have a molecular weight cut-off value (MWCO) of greater than 1,000,000 Dalton, such as greater than 1,200,000 Dalton, e.g. greater than 1,500,000 Dalton. At this point in the process, the dry matter content is typically 1-5%. Advantageously, the pH of the first liquid fraction may be adjusted to a pH between 3 and 5, such as around pH 4, before being processed further.

The first concentrated biomass material typically has a protein content of 50-80% w/w and a nucleic acid content of 0-5% w/w.

With reference to FIG. 1b: the first liquid fraction from the first separation process is then subjected to a second nanofiltration (NF) step, to provide a mineral-rich first NF-permeate, and a nucleic acid and peptide-rich first NF retentate in which the nucleic acids are dissolved. This NF step suitably has a MWCO of between 100-2000 Da.

The nucleic acid and peptide-rich first NF retentate from the second NF step typically has a dry matter content of 3-15%, a protein content of 40-75% w/w and a nucleic acid content of 10-40% w/w.

With reference to FIG. 1d: the first liquid fraction from the first separation process to a second filtration step, to provide a mineral-rich second permeate, and a nucleic acid-rich second retentate in which the nucleic acids are dissolved. The ratio of nucleic acids to minerals in the second retentate is increased with at least 30% compared to the same ratio in the first liquid fraction.

The second separation process may be an ultrafiltration (UF) process or a microfiltration (MF) process, and is preferably an UF process.

The UF membrane is a semi-permeable dynamic disc filter with a pore size of 20 nm or 5000 Da MWCO. This device also includes a backflush system that sends a part of the permeate back to the membrane every 20 sec. In this system, the rotating discs limit the membrane clogging and the formation of a polarisation layer. The UF is performed on a maximal period of 24 hours e.g. for a period less than 24 hours, e.g. for a period less than 15 hours, e.g. for a period less than 11 hours, e.g. for a period less than 8 hours, e.g. for a period less than 6 hours, e.g. for a period less than 4 hours.

### Option 1d-1

In one option, the nucleic acid-rich second retentate from the second filtration step is taken directly for spraydrying. The product is rich in nucleic acids, peptides and minerals.

### Option 1d-2

In another option, the (MF/UF) nucleic acid-rich second retentate from the second filtration step is subjected to a second nanofiltration (NF) step to obtain a nucleic acid rich product (second NF-retentate).

The second nanofiltration (NF) process also provides a mineral-rich second NF permeate. The second NF permeate typically comprises monovalent ions.

The nucleic acid rich second NF-retentate comprises 0.5-10% (w/w) dry matter, preferably 0.5-5% (w/w) dry matter, more preferably 2-3% (w/w) dry matter. This product typically comprises 10-90% (w/w) nucleic acids, preferably 40-80% (w/w) nucleic acids, more preferably 50-70% (w/w) nucleic acids.

The method may further comprise the step of drying, preferably spray-drying, the nucleic acid rich second NF-retentate, to provide a dry nucleic-acid rich product.

The nucleic acid rich product may be used, directly or as further processed according to pending regulations, as an ingredient for food or feed. In particular, the nucleic acid rich product may be used as an ingredient for baby food or infant formula.

### Option 1d-3

In another option the MF/UF mineral-rich second permeate is subjected to a third nanofiltration (NF) process to obtain a RNA rich third NF-retentate and mineral rich third NF-permeate. The third NF permeate typically comprises monovalent ions.

The ratio of RNA to minerals in the third NF-retentate is increased with at least 30% compared to the same ratio in the second permeate.

The RNA rich third NF-retentate comprises 0.5-20% (w/w) dry matter, preferably 0.5-10% (w/w) dry matter, more preferably 6-7% (w/w) dry matter. This product typically comprises 10-90% (w/w) RNA, preferably 40-80% (w/w) RNA, more preferably 50-70% (w/w) RNA.

The method may further comprise the step of drying, preferably spray-drying the RNA rich third NF-retentate, to provide a dry RNA rich product.

The RNA rich product may be used, directly or as further processed according to pending regulations, as an ingredient for food or feed. In particular, the RNA rich product may be used as an ingredient for baby food or infant formula.

### Example 1

The M. capsulatus biomass material can be prepared by the method described in Larsen and Jørgensen, Appl. Microbiol. Biotechnol., 1996. In short, M. capsulatus, e.g., M. capsulatus Bath (NCIMB 11132), is grown in a bioreactor in a suitable medium with methane as the carbon source. The cells are then heat-shocked by heating the cell suspension to 90° C. for 10 seconds, after which the cells are incubated at 60° C. for 20 minutes at pH 7.0.

### LIST OF REFERENCES

J. Larsen & L. Jørgensen Applied Microbiology and Biotechnology volume 45, pages 137-140 (1996)
WO 2018/115042

1. A method for isolating a nucleic acid rich product from an aqueous biomass material, said method comprising the steps of:
subjecting the aqueous biomass material to a heat-shock treatment to provide a heat-shock treated biomass material;
incubating the heat-shock treated biomass material at an elevated temperature for a predetermined time period;
separating the aqueous biomass material in a first separation process, to provide a first concentrated biomass material and a first liquid fraction;
followed by:
subjecting the first liquid fraction from the first separation process to a first nanofiltration (NF) step, to provide a mineral-rich first NF permeate, and a nucleic acid and peptide-rich first NF retentate in which the nucleic acids are dissolved;
or
subjecting the first liquid fraction from the first separation process to a second filtration step, to provide a mineral-rich second permeate, and a nucleic acid-rich second retentate in which the nucleic acids are dissolved;
followed by:
subjecting the nucleic acid-rich second retentate from the second filtration step to spraydrying to obtain a nucleic acid-rich product;
or
subjecting the nucleic acid-rich second retentate from the second filtration step to a second nanofiltration (NF) step, to obtain a nucleic acid and peptide-rich second NF-retentate and a mineral rich second NF-permeate;
or
subjecting the mineral-rich second permeate from the second filtration step to a third nanofiltration step to obtain an RNA rich third NF-retentate and mineral rich third NF-permeate.

2. The method according to aspect 1, further comprising the step of drying, preferably spray-drying, the first NF-retentate, the second NF-retentate and/or the third NF-retentate, to provide a dry nucleic-acid rich product.

3. The method according to any one of the preceding aspects, wherein the first separation process is an ultrafiltration (UF) process, a microfiltration (MF) process or a centrifugation process, preferably a UF process.

4. The method according to any one of the preceding aspects, wherein the second separation process is an ultrafiltration (UF) process, a microfiltration process, or a centrifugation process, and is preferably a UF process.

5. The method according to any one of the preceding aspects, wherein the aqueous biomass material is washed and diluted prior to the first separation process.

6. The method according to any one of the preceding aspects, wherein the heat-shock treatment takes place at a temperature of at least 70° C., preferably at least 80° C. and more preferably at least 90° C.

7. The method according to any one of the preceding aspects, wherein the heat-shock treatment takes place for a time of between 1 and 200 seconds, preferably between 5 and 100 seconds and more preferably between 10 and 50 seconds.

8. The method according to any one of the preceding aspects, wherein incubation of the heat-shock treated biomass material takes place at a temperature of at least 40° C., preferably at least 50° C. and more preferably at least 60° C.

9. The method according to any one of the preceding aspects, wherein incubation of the heat-shock treated biomass material takes place for a time of between 1 and 200 minutes, preferably between 5 and 100 minutes and more preferably between 10 and 50 minutes.

10. The method according to any one of the preceding aspects, wherein the aqueous biomass material is obtained from the fermentation of at least one microorganism, preferably wherein at least one of the microorganisms is a bacterial cell, preferably a methanotroph, more preferably Methylococcus capsulatus.

11. The process according to any one of the preceding aspects, wherein the aqueous biomass material is obtained from the fermentation of a mixture of a methanotrophic bacteria and one or more heterotrophic bacteria.

12. The process according to aspect 11, wherein the heterotrophic bacteria is selected from the group consisting of Ralstonia sp.; Bacillus brevis; Brevibacillus agri; Alcaligenes acidovorans; Aneurinibacillus danicus and Bacillus firmus.

13. The method according to any one of the preceding aspects, wherein the nucleic acid rich product comprises 0.5-10% (w/w) dry matter, preferably 0.5-5% (w/w) dry matter, more preferably 0.5-2% (w/w) dry matter.

14. The method according to aspect 13, wherein the nucleic acid rich product typically comprises 10-90% (w/w) nucleic acids, preferably 40-80% (w/w) nucleic acids, more preferably 50-70% (w/w) nucleic acids.
